(19) United States
(12) Patent Application Publication
(10) Pub. No.: US 2007/0161927 A1
(43) Pub. Date: Jul. 12, 2007

(54) REMINDER SYSTEM AND METHOD FOR URINE COLLECTION

(76) Inventor: John T. Daugirdas, Burr Ridge, IL (US)

Correspondence Address: BROWN & MICHAELS, PC;400 M & T BANK BUILDING, 118 NORTH TIOGA ST, ITHACA, NY, 14850, US

(21) Appl. No.: 11/690,905

(22) Filed: Mar. 26, 2007

Related U.S. Application Data
(63) Continuation-in-part of application No. 10/386,296, filed on Mar. 11, 2003, now Pat. No. 7,211,051.

(51) Int. Cl. A61B 5/00

(52) U.S. Cl. 600/573

(57) ABSTRACT

The method for reminding an individual to collect a urine specimen includes providing at least one reminder label such that the individual will discover the label in the course of preparing to urinate, thereby serving as a reminder to collect the urine specimen, attaching the label to a location where the label will be encountered by the individual when preparing to urinate, discovering the label when preparing to urinate, reminding the individual to collect the urine specimen when the individual discovers the label, and collecting the urine specimen in a collection container. Reminder labels include, but are not limited to, pads, clips, guards, temporary tattoos for use on skin, adhesive paper labels, adhesive plastic labels, adhesive foil labels, and textile labels.

### REFERENCE TO RELATED APPLICATIONS

This is a continuation-in-part patent application of copending application Ser. No. 10/386,296, filed Mar. 11, 2003, entitled “A REMINDER SYSTEM FOR PERSONAL MEDICAL SAMPLING OR TREATMENT PROCEDURES”. The aforementioned application is hereby incorporated herein by reference.

### BACKGROUND OF THE INVENTION

1. Field of the Invention

The invention pertains to the field of information reminding systems. More particularly, the invention relates to the field of urine sample collection reminders.

2. Description of Related Art

An efficacious reminder system is important in certain medical treatments or programs of treatment. This is especially true for the collection of urine specimens.

In medicine, urine is commonly collected for at least four purposes. One purpose is to quantify how much protein is lost in the urine over a 24-hour period. The amount of protein lost directly pertains to whether or not the patient has a serious kidney disease, and 24-hour urine specimens are routinely collected at intervals to monitor the response to treatment of disease that cause protein leakage into the urine.

Another purpose, again utilizing a 24-hour collection time frame, is used to measure the amount of creating in the urine. If the 24-hour amount of creating in the urine is accurately known, and the creating level in the blood also is determined, the functional capacity of the kidney can be determined.

Still another purpose is for patients having renal stones. Urine is collected from these patients to determine the 24-hour excretion rate of various minerals and other chemicals such as uric acid, calcium, sodium, and citrate.

Yet another purpose is to help diagnose patients with endocrine diseases or difficult to treat hypertension. In such patients, a correct diagnosis depends on knowing the 24-hour excretion amount of various hormones, such as aldosterone, various other steroid hormones, or derivatives of catecholamines.

In all of these instances, the common method is to give the patient a large collection bottle, which may or may not be kept refrigerated, that the patient is sent home with. It is the patient's responsibility to collect all of the urine during the 24-hour period into the bottle. However, a patient normally urinates from 4-8 times over a 24-hour period. Often a patient must use different bathrooms, in his or her house, at work, or other places. During the normal stresses of the day, it is quite easy for a patient to simply go to a bathroom and urinate directly into the toilet, forgetting to collect the sample and thereby causing the collection of urine to be incomplete. This has important consequences. In the case of proteinuria, it can cause the amount of protein leakage to be underestimated, which may lead to an inappropriate diagnosis. In the case of creating, the functional capacity of the kidney will be underestimated. In the case of uric acid, calcium, sodium, or citrate, the mineral excretion rate will be underestimated, which may lead to errors in diagnosis and treatment. In the case of endocrine disorders or difficult-to-treat hypertension, if a specimen does not contain a complete urine sample of at least 24 hours, a diagnosis of a potentially treatable endocrine or hypertensive condition can be missed. Collection of urine in children is especially difficult, given their active lifestyles.

The alternative, performing an incomplete urine collection and testing, is by orders of magnitude more costly, since it necessitates repeating every step of the process: another doctor's consultation with a patient; another collection regime requiring another kit; another round of laboratory testing; and, reanalysis of the results, after which the patient may require another consultation. The inconvenience and burden on the treatment provider, the patient, and overall on the health care system, in addition to the immediate monetary costs, should be evident and can prove very significant.

Devices and reminder systems have previously been provided to remind an individual of certain dates or duties. Some of these systems require an individual to provide input or perform a manual operation of some sort, as for example in the use of visual and handwritten appointment books or electronic calendar devices. Other systems may provide visual or audio aids and features to accomplish a reminder function, examples of which include wristwatch beepers and personal pagers or the like.

U.S. Pat. No. 4,820,164 discloses training pants for a toddler having a releasably attachable pad on the front of the pants containing several figures hidden from view by individual releasable patches of fabric. The figures as such provide a visual reward system for a toddler upon removal of a patch and display of a figure.

U.S. Pat. No. 4,423,734 discloses a nursing bra having an integral reminder device that includes a base member on which is mounted a designating member movable between two indicating positions. The nursing reminder device is not designed to impose behavior modification or to change an established pattern of behavior. Breast-feeding will continue whether or not the nursing bra is employed. The nursing bra simply serves as a marker to identify which of two breasts was last used for feeding.

U.S. Pat. No. 4,179,833 discloses a strap-like band having one or more attached, protruding reminder tabs that attaches to a person's wrist and provides a visual reminder to the wearer.

One disadvantage of prior art reminder devices is that some only provide a visual reminder and therefore can easily remain unnoticed or be ignored. Another disadvantage of some prior art reminders is that they are not geared to or suitable for dispensing by primary care providers, such as physicians, physician's assistants, or nurses, to a patient as part of a patient's self-administered specimen collection or treatment kit or activity.

Still another disadvantage of some reminder devices is their relative complexity. This is especially true in regard to electronic devices like electronic handheld calendars/reminders, and is reflected in their relatively higher cost. An additional disadvantage is the requirement that the user input information in order to activate the reminder function. The user must also ensure that the device is working, powered up, not low on charge, and so forth. A disadvantage of watch alarm-type devices is that these provide just a generic reminder without focusing on a specific activity.

### SUMMARY OF THE INVENTION

According to the present invention, there is provided a method for reminding an individual to collect a urine specimen that includes providing at least one reminder label, wherein the individual will discover the label in the course of preparing to urinate, thereby serving as a reminder to collect the urine specimen. The method also includes attaching the label to a location where the label will be encountered by the individual when preparing to urinate and discovering the label when preparing to urinate. The method further includes reminding the individual to collect the urine specimen when the individual discovers the label and collecting the urine specimen in a collection container.

According to another aspect of the invention, a urine collection kit for an individual includes at least one reminder label, wherein the individual will discover the label in the course of preparing to urinate, thereby serving as a reminder to collect a urine specimen.

The invention is convenient and easy to use since it merely requires the user to attach the reminder label to a location in which the label will serve the intended reminder function. In particular, the reminder label system helps ensure completeness of a 24-hour (or other time period) collection of urine, because it places an unaccustomed object, the reminder label, in a place where it will be encountered in the course of normal urination, both for males and females.

The reminder label further has the advantage of being very inexpensive. Labels such as adhesive labels or tattoos can be produced in quantity, and disposable labels can therefore be supplied to the patient along with or as part of a urine collection kit at very low cost.

The reminder label accordingly helps alleviate the collective costs and burdens on the treatment provider, patient, and indeed the health care system as a whole, that as discussed above can be very significant. The invention is a low cost method of fulfilling an important task and serves important diagnostic and therapeutic functions by enabling the timely completion of tests and evaluations that necessitate self-administered sampling or treatment. The invention can provide important health benefits to a patient by helping to eliminate delay in treatment, by expediting prompt diagnosis and follow-up treatment, and by helping to promote faster and better treatment of illnesses or chronic diseases and the like. The invention accordingly serves to promote more efficient health care and reduce overall health care costs.

### BRIEF DESCRIPTION OF THE DRAWINGS

FIG. 1 shows a perspective, partially fragmentary view of an individual with a reminder pad of the present invention (shown in phantom) attached to an undergarment (shown in phantom).

FIG. 2 shows a perspective view of a male wearing a reminder pad in an embodiment of the present invention.

FIG. 3 shows a top plan view of a reminder pad of the present invention.

FIG. 4 shows a perspective view of a reminder clip of the present invention attached to the fly of a male brief.

FIG. 5 shows a front elevational view of an undergarment with attached reminder clip of the present invention.

FIG. 6 shows a perspective view of a female wearing a reminder pad in an embodiment of the present invention.

FIG. 7 shows a reminder label in an embodiment of the present invention.

FIG. 8 shows use of the reminder labels on the body in an embodiment of the present invention.

FIG. 9 shows use of the reminder labels on a toilet in an embodiment of the present invention.

FIG. 10 shows reminder tattoos in an embodiment of the present invention.

FIG. 11A shows a first step of applying a shield of the present invention.

FIG. 11B shows a second step of applying a shield of the present invention.

FIG. 11C shows a third step of applying a shield of the present invention.

FIG. 12 shows a reminder label of the present invention applied to an undergarment.

FIG. 13 shows a different type of reminder label applied to an undergarment.

FIG. 14 shows yet another type of reminder label applied to an undergarment.

FIG. 15 shows a reminder label in the form of a clip that can be applied to either the waistband or to a fly-partition of an undergarment.

### DETAILED DESCRIPTION OF THE INVENTION

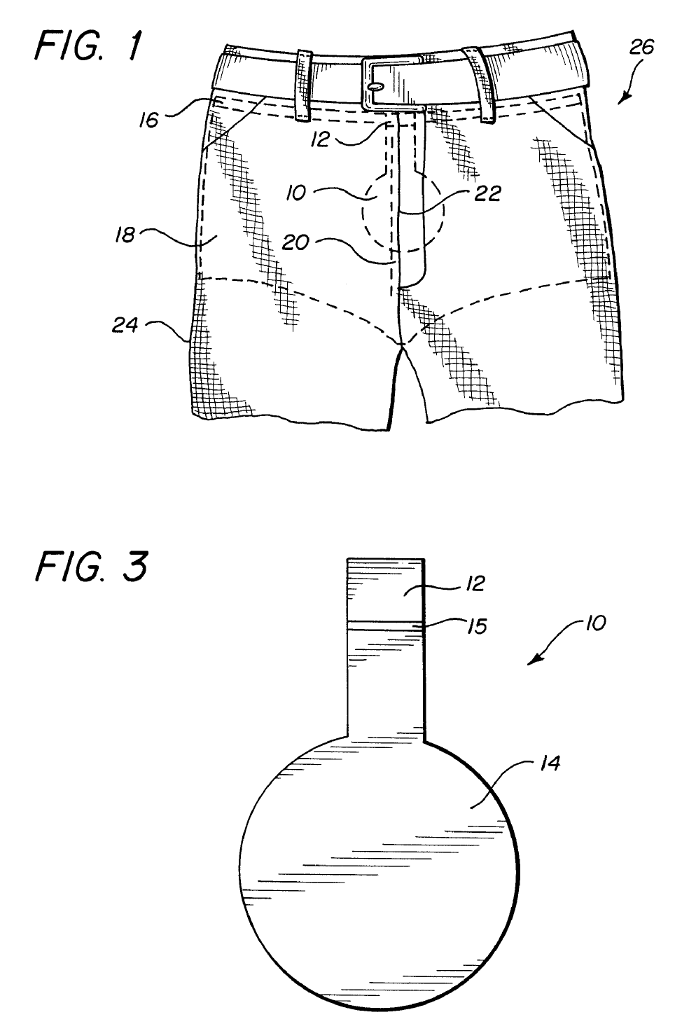

The personal reminder system and method for collecting a bodily fluid specimen or administering a self-treatment can best be understood by reference to the accompanying drawings and as follows. The terms “individual”, “user” and “wearer” in the description that follows are used interchangeably. The terms “self-administer” or “self-treatment” as used herein also include a treatment provider assisting an individual in collecting the specimen or administering the treatment, for example, if a patient is too young, physically handicapped, or for any other reason unable to perform the procedure themselves, as will be made clearer below. The term “brief” as used herein is intended to include both male and female underwear unless noted otherwise. The term “pad” as used herein also includes a clip as is further described below. The term “reminder label” as used herein, includes, but is not limited to, a reminder pad or clip (as shown in FIGS. 1 through 5), a fly guard (shield), a temporary tattoo for use on skin, an adhesive paper label/sticker, an adhesive plastic label/sticker, an adhesive foil label/sticker, and a textile label. The tattoos, adhesive labels and textile labels may be of any shape including, but not limited to, square, rectangular, circular, oblong, oval, or triangular.

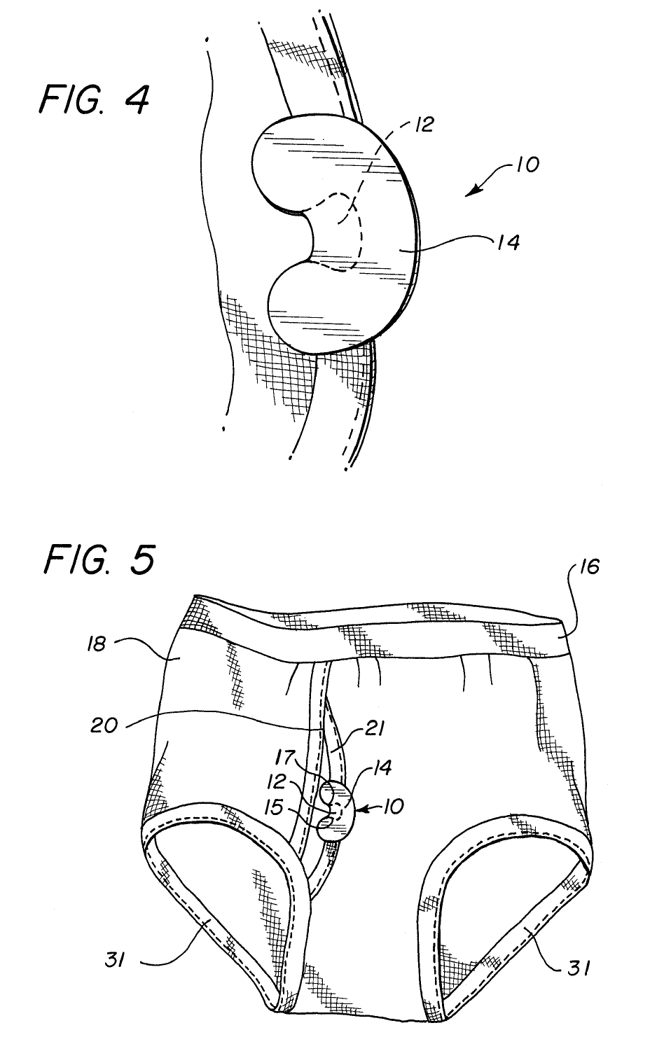

As illustrated in FIGS. 1-5, reminder pad 10 has a foldable portion, tab 12, and reminder area 14, and as shown in FIG. 3 can have a pre-creased, compressed, or weakened area 15 by which tab 12 can be folded back against pad 10. Pad 10 may therefore be attached by tab 12 to waistband 16. In another embodiment illustrated in FIG. 4, reminder area 14 of pad or clip 10 may have flared portions 17 such that when attached by tab 12 to waistband 16 of male brief 18 portions 17 overlie both sides of vertical fold 21 of brief fly 20. Tab 12 may be preformed and integral with pad 10, for example, as with a one-piece, molded plastic, compression-fit type of pad 10. Pad 10 can also comprise a nickel-sized fold-over clip, such as metal or plastic, that could be attached to vertical fold 21 of fly 20.

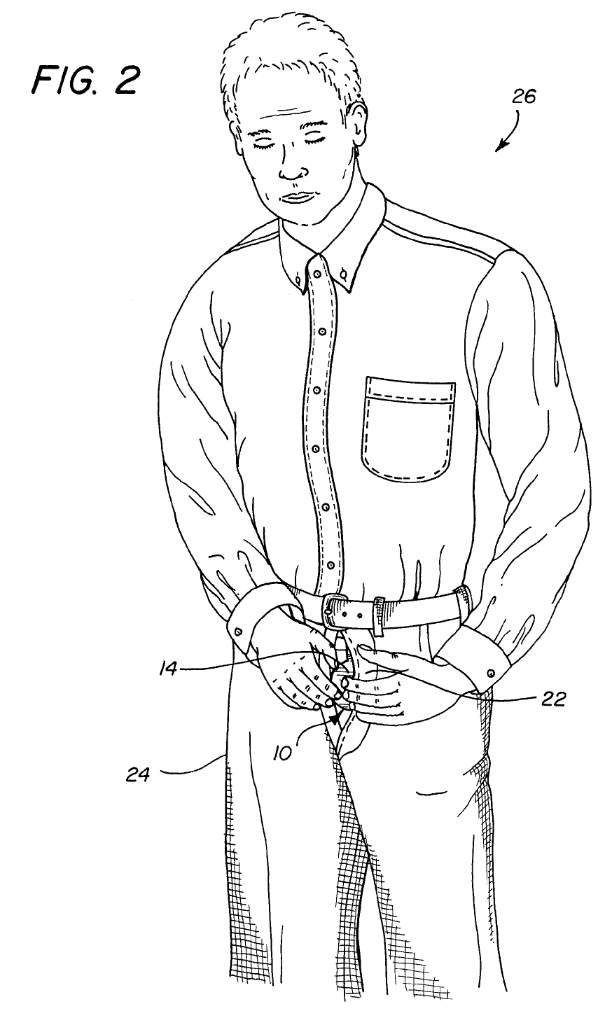

Pad 10 may be positioned either between brief 18 and the wearer's body or between brief 18 and the wearer's outerwear, the latter being illustrated in FIG. 2 where pad 10 is worn between brief 18 and fly 22 of outerwear 24 by male 26. Examples of outerwear 24 for use in the invention for either male or female wearers include but are not limited to trousers, skirts, dresses, pantyhose, or stockings or the like. In any event, pad 10 is hidden from view under outerwear 24 in the practice of the invention.

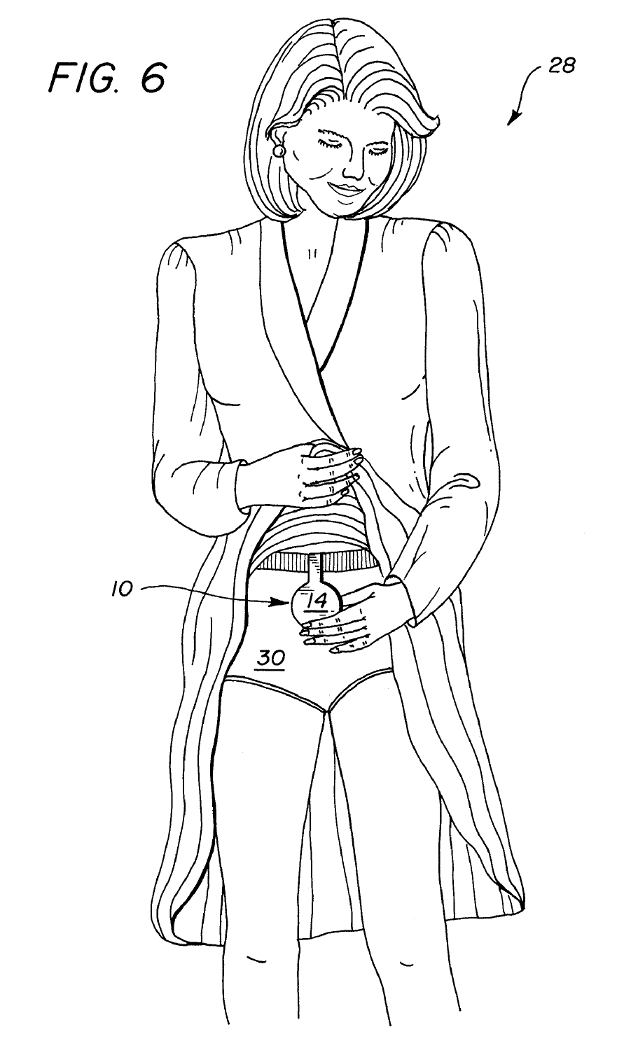

FIG. 6 shows female user 28 attaching pad 10 to flyless female brief 30, and as is described above for a male user, pad 10 is positioned in the area of the groin. Reminder area 14 of pad 10 then serves the function of reminding individual 26 or 28 or a treatment provider to collect a specimen such as a urine sample or to perform a treatment such as to administer a topical agent or some other type of medication to that region of the body.

Pad 10 can be fabricated from any convenient material, the choice of which may depend on its relative cost, durability, workability, and so forth. A preferred such material is cardboard, for example a lightweight cardboard having a thickness in the range of from about 20 mils to about 100 mils.

The size of pad 10 should be such that the wearer will encounter pad 10 and reminder area 14 in the course of performing a normal bodily function such as urinating and thereby serve as a tactile reminder to collect the specimen. Preferably, reminder area 14 has an area in the range of from about ½ to about 10 square inches. FIGS. 1 and 6 show pad 10 suspended from waistband 16 with reminder area 14 of sufficient size so that the male or female wearer will touch pad 10 on either male brief 18 or female brief 30 when preparing to urinate. In these embodiments, reminder area 14 has an area preferably in the range of from about 3 to about 10 square inches. FIG. 3 illustrates this type of pad 10 possessing a tennis racket-shape where substantially circular or oval reminder area 14 tapers into extending rectangular portion 12.

FIG. 5 shows pad 10 attached to vertical fold 21 of fly 20. In this embodiment, remainder area 14 preferably has an area in the range of from about ½ to about 2 square inches. A reminder pad of this size could similarly be attached at an appropriate location on female brief 30, and with either brief 18 or 30 pad 10 could optionally be attached to leg opening 31. When attached to the fly, the wearer upon preparing to urinate could opt to relocate it or push it aside instead of removing pad 10 and then having to reattach it.

Likewise, an appropriately sized and shaped pad 10 could be attached to the back of waistband 16 of brief 18 in the area of the wearer's buttocks, or elsewhere on brief 18, to remind the wearer in the course of performing a normal bodily function such as passing stool that must be collected, for example for a hemoccult, or administer a treatment at or near the site where pad 10 is so positioned.

Pad 10 as discussed above may be a clip, such as a one-piece foldable metal clip similar to a lapel badge or one having a double circle connected by a foldable bridge. Pad 10 may also be a modified safety pin presenting a nickel-size reminder area 14 when worn on brief 18 or 30. In another embodiment, pad 10 may be a metal nose clip on the outside of which is a metallic or plastic sphere serving as reminder area 14.

The invention also includes the method of an individual 26 or 28, or a treatment provider, encountering pad 10, being reminded by the presence of pad 10 to collect a specimen or conduct a treatment, and then collecting the specimen or performing the treatment.

The user, prior to the step of collecting the specimen or performing the treatment, may optionally either remove pad 10, reposition pad 10 as may be convenient, or remove the clothing on which pad 10 is secured.

As shown in FIG. 3, reminder area 14 of pad 10 is circular and as such is configured and sized to cover a sufficient portion of a desired area on a user's clothing or person such that the reminder function is effectuated while any resulting inconvenience or discomfort to the user is minimized. For example, as illustrated in FIG. 1, a male may select pad 10 having a size and shape that covers a substantial portion of fly 20. Pad 10 may also be positioned for convenience or comfort, for example off to one side or even underneath fly 20. The practice of the invention therefore includes the use of pads 10 of varying sizes and shapes, and the selection of the appropriate pad 10 to include in a specific collection or treatment kit or procedure may depend on factors such as the size of the user, the age of the user, and the area where pad 10 will be worn, to name but a few.

As alluded to in the preceding paragraph, the invention further includes providing to a health care provider or patient pad 10 as a component of or addition to a specimen collection or self-administered treatment kit, for example, a urine specimen collection kit or a hemoccult test kit. Thus, a urine collection kit of the present invention may include: a container for holding a collected urine specimen, for example a plastic jug having a capacity of from about one to two liters of fluid; optionally, an anti-bacterial agent or preservative in the container for maintaining the biological efficacy of the collected specimen; optionally, a set of written or printed instructions; and pad 10 which can either be included in the kit or provided separately as an addition to the kit. Although in the figures pad 10 is attached by means of tab 12, other means for attaching or securing pad 10 to brief 18 include an adhesive backing with or without a release sheet, VELCRO brand interlocking tape or the equivalent, buttons, snaps, and other fasteners well known in the art. The invention can also include the use of a separate fastening means, for example a safety pin, paper clip, or binder clip or the like.

The reminder system can serve both to remind the user to collect the sample themselves or if for some reason they are unable to do so then to remind a treatment provider such as a spouse, relative, or nurse, to do so.

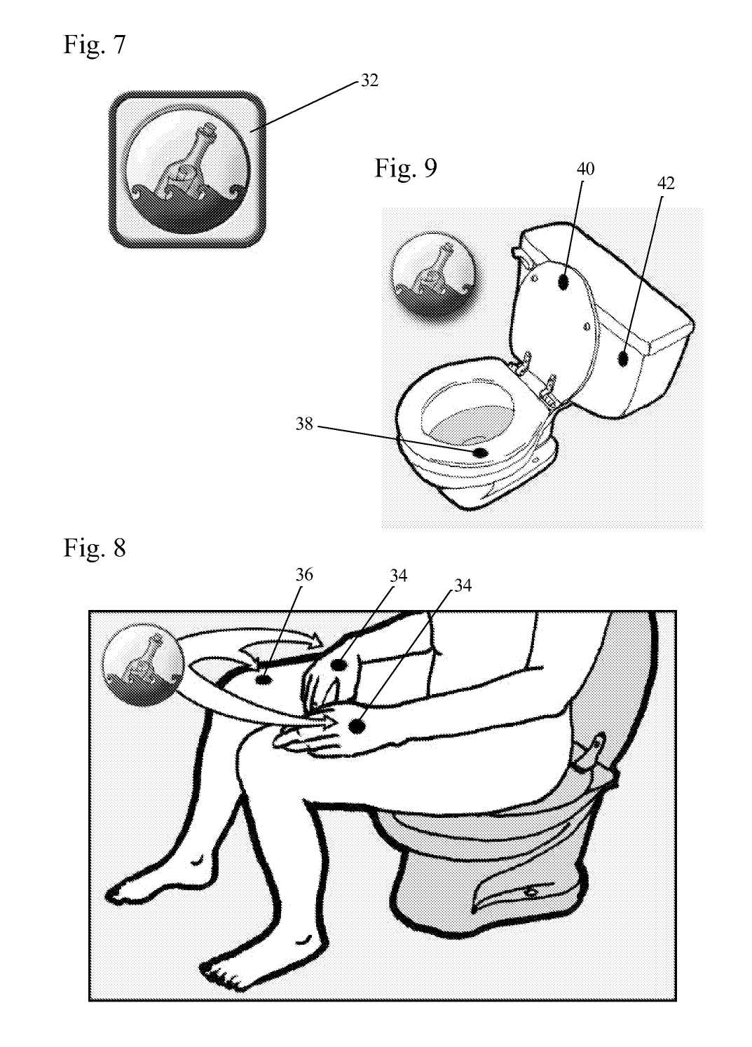

FIGS. 7-15 show alternative reminder systems of the present invention. A doctor may need to know what amount of certain compounds the user is putting out in the urine over a given time-interval. Usually this is over about 24 hours. The start and finish times need to be recorded accurately so the laboratory is able to adjust the result to 24 hours. The test may be ruined if the urine is not properly collected. Reminder labels of the present invention, and kits using the reminder labels, help the user to collect all of his or her urine. This helps the user's doctor to make the proper diagnosis.

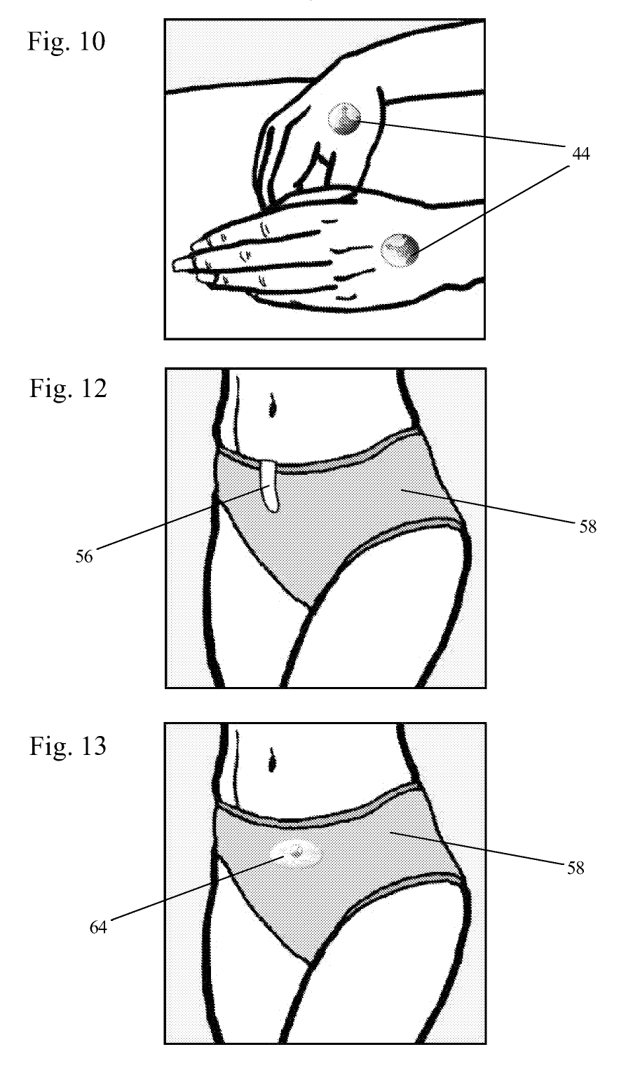

In one embodiment, shown in FIG. 10, the reminder label is a tattoo. The size of the tattoo may be varied, depending upon where the tattoo is going to be placed. For example, the tattoos may be 1.5-inch or 2-inch diameter circular tattoos. The tattoo is not permanent and is preferably designed so that no one knows what it means except the user. The user may apply the tattoo 44 to either the back of the hand, as shown in FIG. 10, or, more discreetly, to the upper thigh area on the front of the leg, so the user sees the tattoo while sitting on a toilet preparing to urinate. A reminder label in the form of a water-based temporary tattoo is preferably applied to the lower thigh or to the back of the hand between the thumb and forefinger, so that it is visible to remind the user when sitting at the toilet and preparing to urinate.

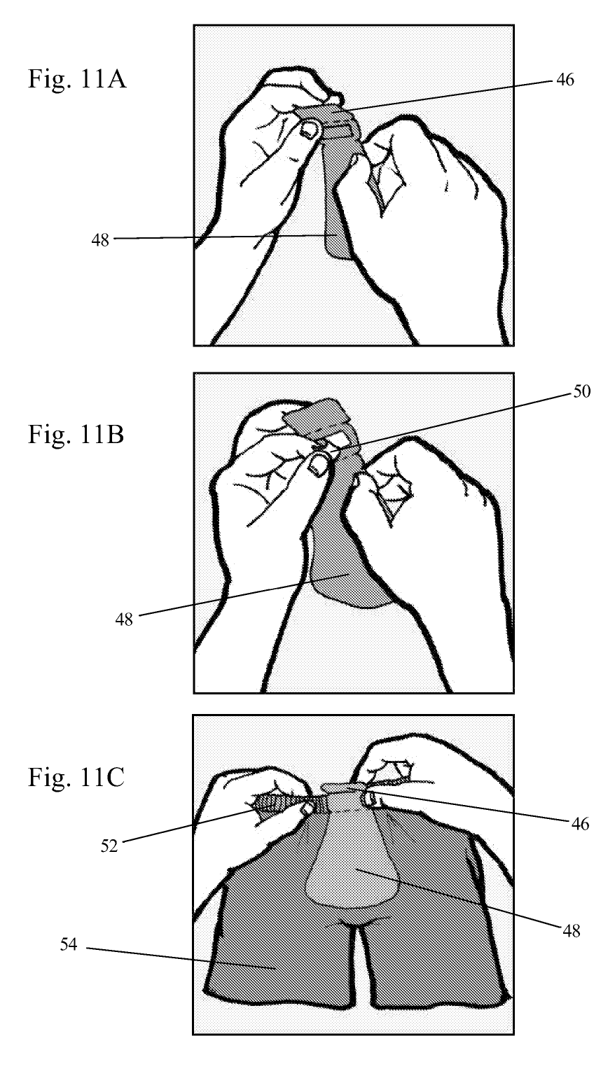

In another embodiment of the present invention, the reminder label is a fly guard or shield, as shown in FIG. 11. In this embodiment, the user bends one section 46 of the shield 48, as shown in FIG. 11A, which folds into the inside of the waistband. The user peels off the backing 50 from an adhesive section, as shown in FIG. 11B. The user folds the tab 46 over the waistband 52 of the undergarment 54 to attach the shield 48 to the undergarment 54, as shown in FIG. 11C. For men, the shield is preferably located over the fly opening so that the fingers touch the area upon opening the pants zipper. The shield is preferably made of Teflon. The shield may be trimmed with scissors if it is otherwise too large for the undergarment, such as women's underwear. The shield is preferably not used when the user is sleeping to avoid accidental dislodgment of the shield.

In yet another embodiment, the reminder labels are adhesive stickers. Oblong reminder labels are preferably attached over the waistband of the undergarment to be a visible reminder at the time of urination. Oval reminder labels 64 are preferably attached to the outside of the undergarment, either in the center for women, as shown in FIG. 13 or to the side of the fly opening for men. The adhesive labels or stickers can be of any size that would be appropriate for the location on which they may be placed. The adhesive labels are also able to adhere to both clothing and hard surfaces.

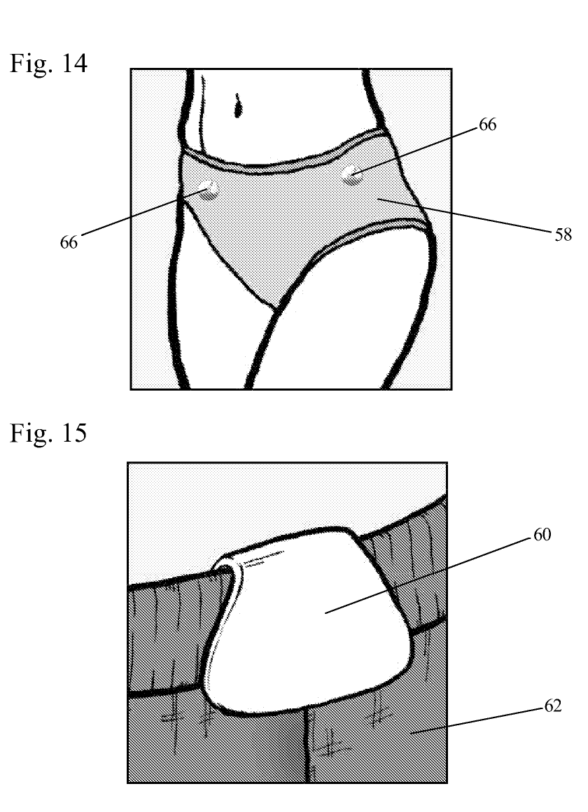

For men, when the reminder label is placed on underwear, the edge of the reminder label is preferably placed next to the fly opening so that the user's fingers touch this area on opening his zipper. For women, the reminder label 64 is put on the middle front of the outside of the user's underwear. Alternatively, for a woman, two labels 66 may be placed on the sides if she touches mostly the sides of her underwear in the process of undressing to urinate. The reminder label 56 may also be folded over the top of the undergarment 58 as shown in FIG. 12. Circular reminder labels are preferably attached to the toilet seat or wherever they may be visible in the bathroom prior to urinating. Circular reminder labels 64 may also be used on the underwear as shown in FIG. 14.

The user may also or alternatively apply adhesive reminder labels to bathroom areas, if the user will be at home for the collection period. These labels are preferably applied to any object that you will encounter in the process of going to the bathroom including, but not limited to, the toilet seat, the toilet tank, the bathroom window and the door handle to the bathroom.

In still another embodiment, a reminder label in the form of a plastic clip 60, as shown in FIG. 15, is preferably attached to the top edge of an undergarment 62. It may be attached simply to the waistband or the fly area for men so that it is encountered in preparing to urinate.

The reminder labels preferably include an image, which helps the user to remember to collect a sample. In one embodiment of the present invention, the image is a message in a bottle, as shown in FIG. 7, but any image that helps an individual remember to collect a urine specimen could be used. The image may be in color or in black and white.

The reminder labels are preferably placed at a location visible to the user when preparing to urinate. Locations include, but are not limited to, the skin, including the back of the hand 34 and the thigh 36 as shown in FIG. 8, the toilet seat 38, the lid of the toilet 40, and the toilet tank 42 as shown in FIG. 9, and a wall near the toilet, a door near the toilet, and a window near the toilet.

In a preferred embodiment, the reminder system is provided to the patient in the form of a kit. The urine collection reminder kit preferably includes multiple labels. These labels are preferably of more than one type and more than one size, to accommodate the needs of different individuals. In fact, any combination of tattoos, pads, fly guards, clips, adhesive labels or textile labels could be used in the kit. The reminder kit also preferably includes pictorial and written directions for use.

As an example, the urine collection reminder kit of the present invention may include:
2 large (2-inch diameter) temporary tattoos
2 small (1.5-inch diameter) temporary tattoos
1 large (3-inch by 5-inch) oval sticker
1 medium (2-inch by 3-inch) oval sticker
1 small (1-inch by 2-inch) oval sticker
1 self-adhesive, textile fly guard
artwork and directions for use

In this example, the oval stickers are preferably removable and preferably Flexo-printed in a four-color process on white gloss paper to work on both clothing and hard surfaces.

A kit of the present invention including reminder labels is preferably used in at least one of the following procedures:

The user preferably urinates into the toilet when first getting up and records this as the start time. The user may write the start time on a collection bottle label and stick the label onto the bottle.

The user preferably gets dressed and applies a reminder label of the present invention to his or her undergarment. In one embodiment, the reminder label is an adhesive sticker or another adhesive label, which preferably goes on the outside of the underwear, such that it is encountered when preparing to go to the bathroom. This triggers the reminder to urinate into the bottle and not the toilet.

Every time the user urinates or has a bowel movement, the user is reminded to collect all of his or her urine and place it in the collection bottle. For some tests the bottle may need to be kept in the refrigerator during the collection period.

In one embodiment, after undressing before bedtime, the user applies a reminder label of the present invention to the outside of the pajamas. The user may also keep the collection bottle by the toilet, if it does not need to be kept refrigerated. Also, the user may stick a reminder label next to the toilet or sink where the user will see the patch before urinating.

It is very important for the user to urinate soon after getting up the following morning and to put this morning urine into the collection bottle. Even though there may not be much of it, this morning urine contains a lot of the compounds that the doctor is interested in measuring. After urinating into the bottle, the user is done and records the time as the finish time.

The user preferably records both the start and finish times on a bottle label and attaches this to the collection bottle. The user removes all of the reminder labels to avoid having them stick too tightly to wherever they have been placed. The user returns the collection bottle to the doctor's office or mails it to the lab per instructions.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method for reminding an individual to collect a urine specimen, comprising the steps of:
a) providing at least one reminder label, wherein the individual will discover the label in the course of preparing to urinate, thereby serving as a reminder to collect the urine specimen;
b) attaching the label to a location where the label will be encountered by the individual when preparing to urinate;
c) discovering the label when preparing to urinate;
d) reminding the individual to collect the urine specimen when the individual discovers the label; and
e) collecting the urine specimen in a collection container.

2. The method of claim 1 further comprising the step of providing the label as part of a urine collection kit to a health care provider or to a patient.

3. The method of claim 2, wherein the urine collection kit comprises a plurality of reminder labels.

4. The method of claim 3, wherein the reminder labels are selected from the group consisting of:
a) at least one reminder pad;
b) at least one reminder clip;
c) at least one fly guard;
d) at least one temporary tattoo for use on skin;
e) at least one adhesive paper label;
f) at least one adhesive plastic label;
g) at least one adhesive foil label;
h) at least one textile label; and
i) any combination of a) through h).

5. The method of claim 1, wherein the location is selected from the group consisting of:
a) skin of the individual;
b) a surface of the toilet;
c) a wall near a toilet;
d) a door near the toilet;
e) a window near the toilet;
f) an undergarment of the individual; and
g) any combination of a) through f).

6. The method of claim 5 further comprising the step of providing the label as part of a urine collection kit to a health care provider or to a patient.

7. The method of claim 6, wherein the urine collection kit comprises a plurality of reminder labels.

8. The method of claim 7, wherein the reminder labels are selected from the group consisting of:
a) at least one reminder pad;
b) at least one reminder clip;
c) at least one fly guard;
d) at least one temporary tattoo for use on skin;
e) at least one adhesive paper label;
f) at least one adhesive plastic label;
g) at least one adhesive foil label;
h) at least one textile label; and
i) any combination of a) through h).

9. A urine collection kit for an individual, the kit comprising:
at least one reminder label, wherein the individual will discover the label in the course of preparing to urinate, thereby serving as a reminder to collect a urine specimen.

10. The urine collection kit of claim 9 further comprising at least one collection container for holding a collected urine specimen.

11. The urine collection kit of claim 10 further comprising a set of directions for use of the kit, the directions comprising an explanation to attach the label to a location where the label will be encountered by the individual when preparing to urinate.

12. The urine collection kit of claim 11, wherein the urine collection kit comprises a plurality of reminder labels.

13. The urine collection kit of claim 12, wherein the reminder labels are selected from the group consisting of:
a) at least one reminder pad;
b) at least one reminder clip;
c) at least one fly guard;
d) at least one temporary tattoo for use on skin;
e) at least one adhesive paper label;
f) at least one adhesive plastic label;
g) at least one adhesive foil label;
h) at least one textile label; and
i) any combination of a) through h).

14. The urine collection kit of claim 12, wherein the location is selected from the group consisting of:
a) skin of the individual;
b) a surface of the toilet;
c) a wall near a toilet;
d) a door near the toilet;
e) a window near the toilet;
f) an undergarment worn by the individual; and
g) any combination of a) through f).

15. The urine collection kit of claim 9 further comprising a set of directions for use of the kit, the directions comprising an explanation to attach the label to a location where the label will be encountered by the individual when preparing to urinate.

16. The urine collection kit of claim 15, wherein the urine collection kit comprises a plurality of reminder labels.

17. The urine collection kit of claim 9, wherein the urine collection kit comprises a plurality of reminder labels.

18. The urine collection kit of claim 9, wherein the reminder labels are selected from the group consisting of:
a) at least one reminder pad;
b) at least one reminder clip;
c) at least one fly guard;
d) at least one temporary tattoo for use on skin;
e) at least one adhesive paper label;
f) at least one adhesive plastic label;
g) at least one adhesive foil label;
h) at least one textile label; and
i) any combination of a) through h).

19. The urine collection kit of claim 9, wherein the location is selected from the group consisting of:
a) skin of the individual;
b) a surface of the toilet;
c) a wall near a toilet;
d) a door near the toilet;
e) a window near the toilet;
f) an undergarment worn by the individual; and
g) any combination of a) through f).

20. The urine collection kit of claim 9, wherein the urine collection kit is supplied to a health care provider or to a patient.
